(19) United States
(12) Patent Application Publication
(10) Pub. No.: US 2022/0313479 A1
(43) Pub. Date: Oct. 6, 2022

(54) HAND-SHAPED STRAPPED ICE PACK APPARATUS

(71) Applicant: Jonathan Bain, Nashville, TN (US)

(72) Inventor: Jonathan Bain, Nashville, TN (US)

(21) Appl. No.: 17/219,098

(22) Filed: Mar. 31, 2021

(51) Int. Cl. A61F 7/02

(52) U.S. Cl. CPC A61F 7/02; A61F 2007/0228; A61F 2007/0231; A61F 2007/0233; A61F 2007/0219

(57) ABSTRACT

A hand-shaped strapped ice pack apparatus for first aid and novelty includes a glove body having a dorsal side, a ventral side, and a sealed bottom side to form an inner cavity. The glove body has a wrist portion, a palm portion, and a finger portion. A gel pack is coupled within the inner cavity and has a specific heat configured to function as an ice pack or a heat pack. A pair of second hook and loop fasteners is coupled to an inside of a strap adjacent a first end and a second end. The pair of second hook and loop fasteners is selectively engageable with a plurality of first hook and loop fasteners coupled to the dorsal side of the glove body. The strap is configured to secure the glove body to a user's body.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

### (2) Description of Related Art Including Information Disclosed Under 37 CFR 1.97 and 1.98

The disclosure and prior art relates to ice packs and more particularly pertains to a new ice pack for first aid and novelty.

### BRIEF SUMMARY OF THE INVENTION

An embodiment of the disclosure meets the needs presented above by generally comprising a glove body having a dorsal side, a ventral side, and a sealed bottom side to form an inner cavity. The glove body has a wrist portion, a palm portion, and a finger portion. A gel pack is coupled to the glove body within the inner cavity. The gel pack has a specific heat configured to function as an ice pack or a heat pack. A plurality of first hook and loop fasteners is coupled to the dorsal side of the glove body. A strap has a first end, a second end, an outside, and an inside. A pair of second hook and loop fasteners is coupled to the inside of the strap adjacent the first end and the second end. The pair of second hook and loop fasteners is selectively engageable with the plurality of first hook and loop fasteners. The strap is configured to secure the glove body to a user's body.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a hand-shaped strapped ice pack apparatus according to an embodiment of the disclosure.

FIG. 2 is an isometric view of an embodiment of the disclosure.

FIG. 3 is a side elevation view of an embodiment of the disclosure.

FIG. 4 is a cross-sectional view of an embodiment of the disclosure along line 4-4 of FIG. 2.

FIG. 5 is a rear elevation view of an embodiment of the disclosure.

FIG. 6 is an in-use view of an embodiment of the disclosure.

### DETAILED DESCRIPTION OF THE INVENTION

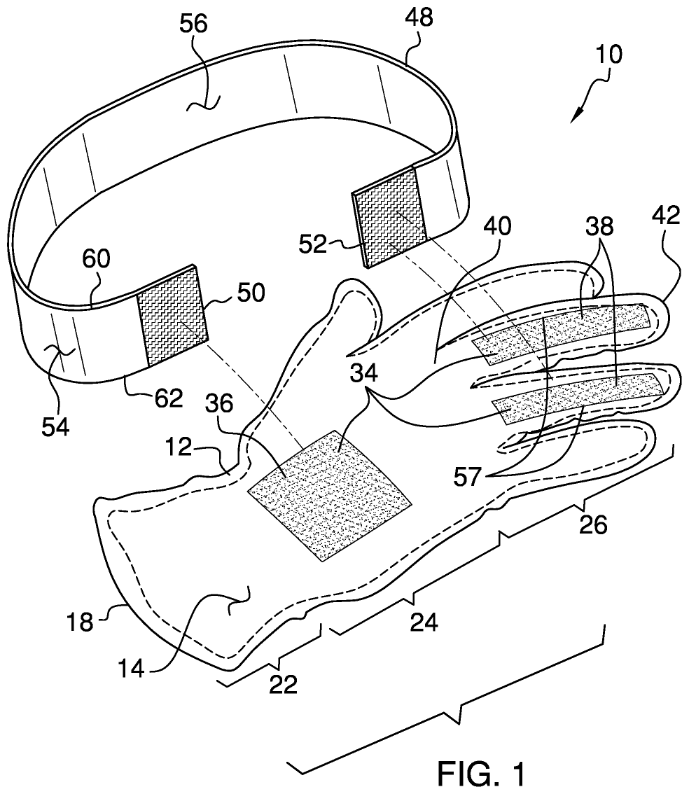

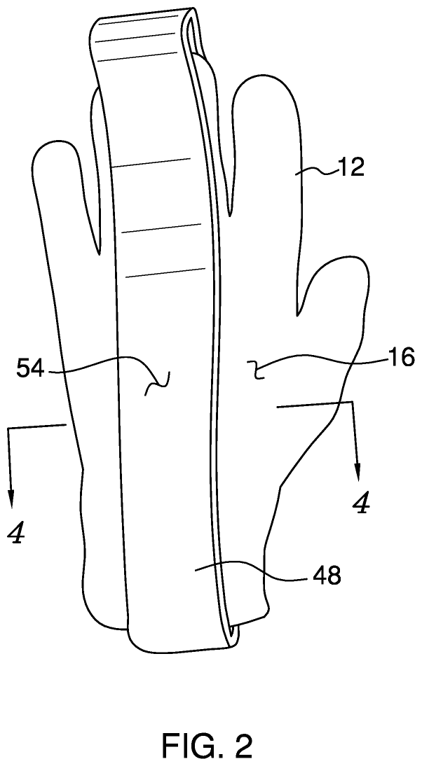

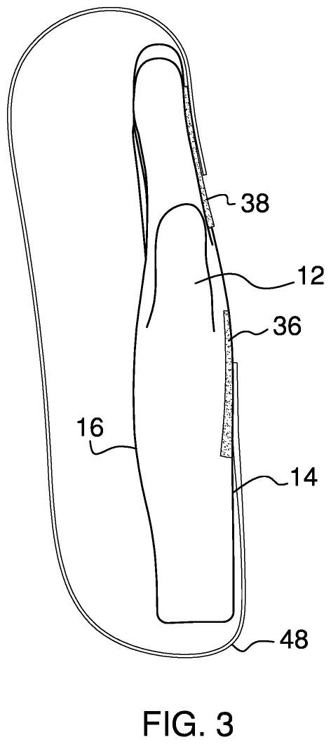

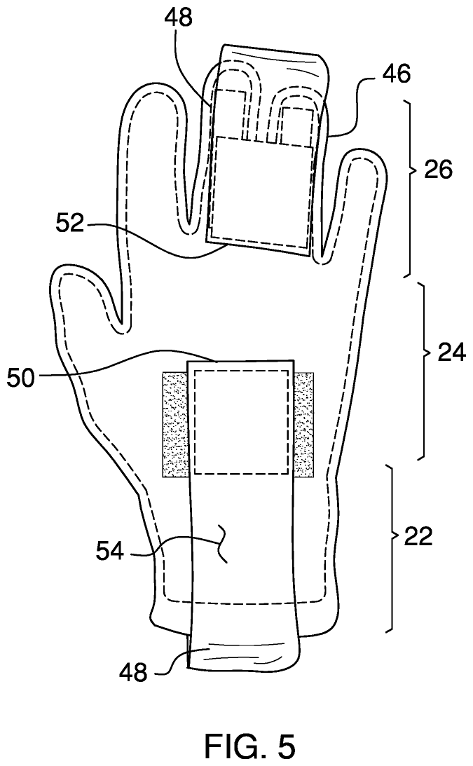

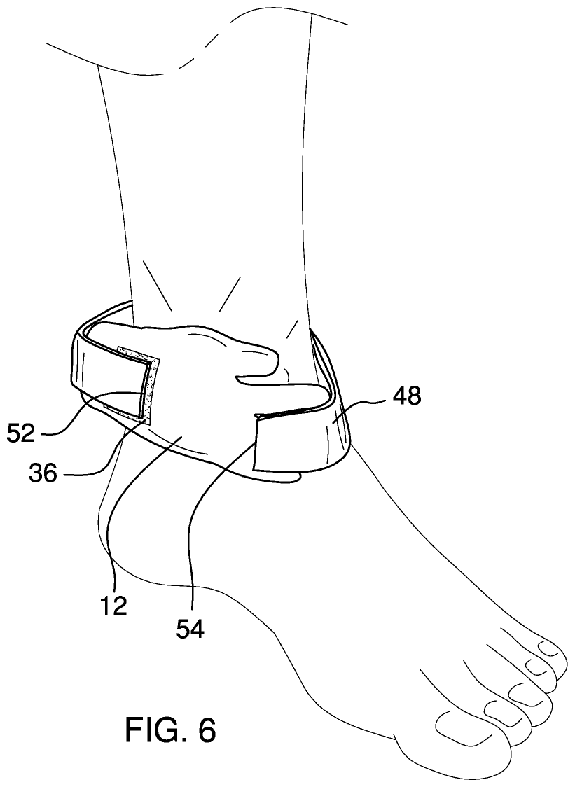

With reference now to the drawings, and in particular to FIGS. 1 through 6 thereof, a new ice pack embodying the principles and concepts of an embodiment of the disclosure and generally designated by the reference numeral 10 will be described.

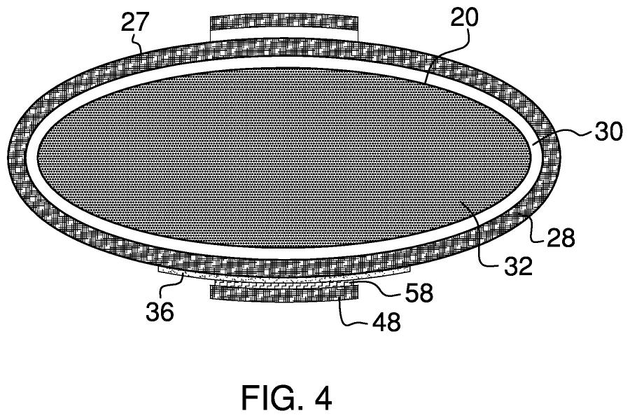

As best illustrated in FIGS. 1 through 6, the hand-shaped strapped ice pack apparatus 10 generally comprises a glove body 12 having a dorsal side 14, a ventral side 16 and a sealed bottom side 18 to form a hand-shaped inner cavity 20. The glove body 12 has a wrist portion 22, a palm portion 24, and a finger portion 26. A cross-section 27 of the wrist portion 22 of the glove body is elliptical when fully expanded (as shown in FIG. 4), yet the glove body 12 may be malleable to mold to the contours of a user's body under applied pressure. The glove body 12 comprises an outer layer 28 and an inner layer 30. The outer layer 28 may be rubberized or plastic. The inner layer 30 is nonpermeable and continuously disposed inside the outer layer 28. A gel pack 32 is coupled to the glove body within the inner cavity 20 and is sealed by the inner layer 30. The gel pack 32 has a specific heat configured to function as an ice pack or a heat pack. Each of the gel pack 32, the inner layer 30, and the outer layer 28 is microwavable.

A plurality of first hook and loop fasteners 34 is coupled to the dorsal side 14 of the glove body. The plurality of first hook and loop fasteners 34 comprises a palm fastener 36 coupled to the palm portion 24 of the glove body and a pair of finger fasteners 38 coupled to the finger portion 26 of the glove body. Each of the pair of finger fasteners 38 is an elongated rectangle extending from adjacent a knuckle side 40 of the finger portion to proximal a tip side 42 of the finger portion. The pair of finger fasteners 38 is coupled to a middle finger 44 and a ring finger 46 of the finger portion.

A strap 48 has a first end 50, a second end 52, an outside 54, and an inside 56. A width of the strap 48 is less than a width of the wrist portion 22 of the glove body and may be equal to a width between a pair of outer edges 57 of the pair of finger fasteners 38. A width of the palm fastener 36 is greater than the width of the strap 48. A pair of second hook and loop fasteners 58 is coupled to the inside 56 of the strap adjacent the first end 50 and the second end 52. Each of the pair of second hook and loop fasteners 58 is rectangular and extends from a top edge 60 to a bottom edge 62 of the strap. The pair of second hook and loop fasteners 58 is selectively engageable with the plurality of first hook and loop fasteners 34. The strap 48 is thus configured to secure the glove body 12 to a user's body.

In use, the glove body 12 is cooled in a freezer or heated in a microwave or a pot of hot water to achieve a desired temperature of the gel pack 32. The second hook and loop fastener 58 of the first end 50 of the strap is then engaged with the palm fastener 36 and the glove body 12 is placed on the area of injury, such as an ankle or a wrist. The strap 48 is then wrapped around the user's body and the second hook and loop fastener 58 of the second end 52 is engaged with the pair of finger fasteners 38 to secure the glove body 12 in place. The glove body 12 may alternatively be used without the strap 48 and held in place or used in a cooler as a novelty ice pack to maintain food temperature.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A hand-shaped strapped ice pack apparatus comprising:
a glove body, the glove body having a dorsal side, a ventral side and a sealed bottom side to form an inner cavity, the glove body having a wrist portion, a palm portion, and a finger portion;
a gel pack coupled to the glove body, the gel pack being coupled within the inner cavity, the gel pack having a specific heat configured to function as an ice pack or a heat pack;
a plurality of first hook and loop fasteners coupled to the dorsal side of the glove body;
a strap having a first end, a second end, an outside, and an inside; and
a pair of second hook and loop fasteners coupled to the inside of the strap adjacent the first end and the second end, the pair of second hook and loop fasteners being selectively engageable with the plurality of first hook and loop fasteners;
wherein the strap being configured to secure the glove body to a user's body.

2. The hand-shaped strapped ice pack apparatus of claim 1 further comprising the plurality of first hook and loop fasteners comprising a palm fastener coupled to the palm portion of the glove body and a pair of finger fasteners coupled to the finger portion of the glove body.

3. The hand-shaped strapped ice pack apparatus of claim 2 further comprising the pair of finger fasteners being elongated rectangles.

4. The hand-shaped strapped ice pack apparatus of claim 3 further comprising the pair of finger fasteners extending from adjacent a knuckle side of the finger portion to proximal a tip side of the finger portion.

5. The hand-shaped strapped ice pack apparatus of claim 2 further comprising the pair of finger fasteners being coupled to a middle finger and a ring finger of the finger portion of the glove body.

6. The hand-shaped strapped ice pack apparatus of claim 1 further comprising the pair of second hook and loop fasteners being rectangular and extending from a top edge to a bottom edge of the strap.

7. The hand-shaped strapped ice pack apparatus of claim 1 further comprising a cross-section of the wrist portion of the glove body being elliptical.

8. The hand-shaped strapped ice pack apparatus of claim 1 further comprising the glove body comprising an outer layer and an inner layer, the inner layer being continuously disposed inside the outer layer to provide a nonpermeable shell for the gel pack.

9. The hand-shaped strapped ice pack apparatus of claim 1 further comprising the inner cavity and the gel pack being hand-shaped.

10. The hand-shaped strapped ice pack apparatus of claim 1 further comprising a width of the strap being less than a width of the wrist portion of the glove body.

11. A hand-shaped strapped ice pack apparatus comprising:
a glove body, the glove body having a dorsal side, a ventral side and a sealed bottom side to form a hand-shaped inner cavity, the glove body having a wrist portion, a palm portion, and a finger portion, a cross-section of the wrist portion of the glove body being elliptical, the glove body comprising an outer layer and an inner layer, the inner layer being continuously disposed inside the outer layer and nonpermeable;
a gel pack coupled to the glove body, the gel pack being coupled within the inner cavity, the gel pack having a specific heat configured to function as an ice pack or a heat pack, the gel pack being sealed by the inner layer;
a plurality of first hook and loop fasteners coupled to the dorsal side of the glove body, the plurality of first hook and loop fasteners comprising a palm fastener coupled to the palm portion of the glove body and a pair of finger fasteners coupled to the finger portion of the glove body, the pair of finger fasteners being elongated rectangles extending from adjacent a knuckle side of the finger portion to proximal a tip side of the finger portion, the pair of finger fasteners being coupled to a middle finger and a ring finger of the finger portion;
a strap having a first end, a second end, an outside, and an inside, a width of the strap being less than a width of the wrist portion of the glove body; and
a pair of second hook and loop fasteners coupled to the inside of the strap adjacent the first end and the second end, the pair of second hook and loop fasteners being rectangular and extending from a top edge to a bottom edge of the strap, the pair of second hook and loop fasteners being selectively engageable with the plurality of first hook and loop fasteners; and
wherein the strap is configured to secure the glove body to a user's body.
